(19) United States
(12) Patent Application Publication
(10) Pub. No.: US 2021/0142504 A1
(43) Pub. Date: May 13, 2021

(54) DETECTION OF FIDUCIALS IN A CLINICAL IMAGE

(71) Applicant: St. Jude Medical International Holding S.á r.l., Luxembourg (LU)

(72) Inventors: Lorina Dascal, Haifa (IL); Itai Winkler, Nofit (IL)

(21) Appl. No.: 16/491,564

(22) PCT Filed: Mar. 8, 2018
(86) PCT No.: PCT/IB2018/051527

§ 371 Date: Sep. 5, 2019

Related U.S. Application Data
(60) Provisional application No. 62/469,423, filed on Mar. 9, 2017.

(51) Int. Cl. G06T 7/70; A61B 90/00; A61B 34/20; G06T 5/20; G06T 5/00; G06T 11/00; G06T 7/00

(52) U.S. Cl. CPC G06T 7/70; A61B 90/39; A61B 34/20; A61M 2025/0166; G06T 5/002; G06T 11/00; G06T 7/0012; G06T 5/20

(57) ABSTRACT

The instant disclosure relates to systems and methods for identifying fiducial markers on one or more images of an anatomical region of a patient. Prior to the identification of the fiducial markers, other objects are identified and removed. In particular, some embodiments are directed toward the detection of fiducial markers in the presence of catheters and other medical devices in a fluoroscopic image. In such an embodiment, identifying and removing the medical devices from the clinical image aids in properly identifying the fiducial markers.

### CROSS-REFERENCE TO RELATED APPLICATION

This application claims priority to U.S. provisional application no. 62/469,423, filed 9 Mar. 2017, which is hereby incorporated by reference as though fully set forth herein.

### BACKGROUND

### a. Field

This invention relates to medical systems such as medical device navigation systems. Specifically, the instant disclosure relates to a system for registering one or more images of an anatomical region of a patient in a coordinate system of a medical system such as a medical device navigation system.

### b. Background Art

It is desirable to track the position of medical devices as they are moved within a body so that, for example, drugs and other forms of treatment are administered at the proper location and medical procedures can be completed more efficiently and safely. One conventional means to track the position of medical devices within the body is fluoroscopic imaging. Fluoroscopy may be undesirable in some applications, however, because it subjects the patient and clinician to high levels of electromagnetic radiation. As a result, alternative medical device navigation systems have been developed to track the position of medical devices within the body. These systems typically rely on the generation of electrical or magnetic fields and the detection of induced voltages and currents on position sensors attached to the medical device and/or external to the body. The information derived from these systems is then provided to a clinician through, for example, a visual display. Oftentimes, a representation of the medical device is displayed relative to a computer model or one or more images (including, but not limited to, fluoroscopic images) of the anatomical region in which the device is being maneuvered. In order to display the medical device at the correct location relative to the model or image, the model or image may be registered within the coordinate system of the navigation system.

Images may be registered in the coordinate system of a medical device navigation system in a variety of ways. The imaging system used to capture the images may be physically integrated with the navigation system, such as the MediGuide™ system sold by St. Jude Medical, or as described in commonly assigned U.S. patent application Ser. No. 11/971,004, published as US 2008/0183071, the entire disclosures of which are incorporated herein by reference. Where the imaging system used to capture the images is physically integrated with the navigation system, the imaging system can be registered with the navigation system during installation and the spatial relationship of the navigation system to the imaging system is thereafter constant and known, obviating the need for registration during each new procedure. Where the navigation system and imaging system are physically separate, however, the changing spatial relationship of the systems makes registration more complicated. One solution is to place a plurality of radiopaque fiducial markers in the field of view of the imaging system at locations known relative to the coordinate system of the navigation system. Because the markers are visible in images produced by the imaging system, the images can be registered with the coordinate system by reconciling the visible location of each marker in the images with that marker's known location in the navigation coordinate system. One drawback to this solution, however, is that the fiducial markers are typically visible in the images seen by the clinician performing the procedure, potentially interfering with the clinician's view of the anatomical region being imaged.

The inventors herein have recognized a need for a system and method for registering a group of images of an anatomical region of a patient in a coordinate system of a medical device navigation system that will minimize and/or eliminate one or more of the above-identified deficiencies. The foregoing discussion is intended only to illustrate the present field and should not be taken as a disavowal of claim scope.

### BRIEF SUMMARY

The instant disclosure relates to systems and methods for registering one or more images of an anatomical region of a patient in a coordinate system of a medical system, such as a medical device navigation system; in particular, the detection of fiducial markers in the presence of catheters and other medical devices in the one or more images.

Aspects of the present disclosure are directed to systems and methods that facilitate the registration of images in a coordinate system of a physically separate medical system, such as a medical device navigation system, using objects such as fiducial markers. As a result, a clinician is presented with a more accurate and detailed image of the anatomical region and related items of interest.

Some embodiments of the present disclosure are directed to a method for identifying and locating fiducial markers on a clinical image. The method includes receiving the clinical image from an imaging system, processing the clinical image to remove false fiducial markers, and identifying fiducial markers in the clinical image. In some more specific embodiments, the method further includes applying an enhancing filter to the clinical image to enhance the appearance of the fiducial markers.

Aspects of the present disclosure are directed to a method to detect medical device electrodes within a clinical image. The method includes receiving the clinical image from an imaging system, applying an enhancing filter to the clinical image to enhance the appearance of the electrodes, removing a background of the clinical image, and identifying electrodes within the clinical image. In more detailed embodiments, the step of identifying electrodes within the clinical image includes adaptive thresholding, component filtering based on size and shape, and extraction of the electrodes.

Other embodiments of the present disclosure are directed to a method for superimposing location data of a medical device from a navigation system onto a clinical image. The method includes: receiving the clinical image from a clinical imaging system; processing the clinical image to remove false fiducial markers; identifying fiducial markers within the clinical image; creating a transformation model that reconciles a first coordinate system of the navigation system with a second coordinate system of the clinical imaging system; determining a location of the medical device in the first coordinate system; applying the transformation model to the location of the medical device in the first coordinate system to determine the location of the medical device in the second coordinate system; and superimposing an image of the medical device onto the clinical image based on the known location of the medical device in the second coordinate system.

Yet further embodiments are directed to a navigation system for a cardiovascular catheter. The navigation system includes a cardiovascular catheter, an optic-magnetic registration plate, a clinical imaging system, a catheter localization system, and controller circuitry. The cardiovascular catheter includes one or more electrodes positioned near a distal tip of the catheter. The electrodes facilitate localization of the distal tip of the catheter. The optic-magnetic registration plate includes a plurality of fiducial markers. The clinical imaging system exposes a clinical image including a patient, the fiducial markers, and the cardiovascular catheter in a first coordinate system. The catheter localization system detects the position and orientation of the one or more electrodes in a second coordinate system. The controller circuitry is communicatively coupled to the clinical imaging system and the catheter localization system. The controller circuitry, based on known locations of the fiducial markers in the second coordinate system and the location of the fiducial markers within the clinical image, determines a transformation model between the first and second coordinate systems. Based on the transformation model, the controller circuitry determines the positions of the electrodes within the first coordinate system. In more specific embodiments, the navigation system may include a display, where the controller circuitry is communicatively coupled to the display and further generates a signal for the display including the clinical image superimposed with a representative image of the catheter based on the determined positions of the electrodes in the first coordinate system.

The foregoing and other aspects, features, details, utilities, and advantages of the present disclosure will be apparent from reading the following description and claims, and from reviewing the accompanying drawings.

### BRIEF DESCRIPTION OF THE DRAWINGS

Various example embodiments may be more completely understood in consideration of the following detailed description in connection with the accompanying drawings, in which:

FIG. 1A is a diagrammatic view of one embodiment of a system for registering a group of images in a coordinate system of a medical system, consistent with various embodiments of the present disclosure.

FIG. 1B is a diagrammatic view of another embodiment of a system for registering a group of images in a coordinate system of a medical system, consistent with various embodiments of the present disclosure.

FIG. 1C is a flow diagram illustrating another embodiment of a method for registering a group of images in a coordinate system of a medical system, consistent with various embodiments of the present disclosure.

FIG. 1D is a flow diagram for fiducial detection and background filtering, consistent with various embodiments of the present disclosure.

FIG. 2A is a fluoroscopic image of a patient's chest with 3 minimally invasive diagnostic/therapeutic catheters therein, consistent with various embodiments of the present disclosure.

FIG. 2B is a filtered version of the fluoroscopic image of FIG. 2A where fiducials in the image have been enhanced, consistent with various embodiments of the present disclosure.

FIG. 3 is a fluoroscopic image of a patient's chest with minimally invasive diagnostic/therapeutic catheters therein, the fluoroscopic image is filtered with a non-linear diffusion-reaction filter to remove a background image, consistent with various embodiments of the present disclosure.

FIG. 4 is a fluoroscopic image of a patient's chest with minimally invasive diagnostic/therapeutic catheters therein, the fluoroscopic image is filtered with a Hessian filter to enhance the fiducials in the image, consistent with various embodiments of the present disclosure.

FIG. 5 is a fluoroscopic image of FIG. 4 with the clustered fiducial elements highlighted and the background suppressed using geodesic distance, consistent with various embodiments of the present disclosure.

FIG. 6A is a fluoroscopic image of a patient's chest with minimally invasive diagnostic/therapeutic catheters therein and wire-bundles of a magnetic localization system and a patient reference sensor on the patient's chest, consistent with various embodiments of the present disclosure.

FIG. 6B is the fluoroscopic image of FIG. 6A with a Hessian filter applied after adaptive thresholding, consistent with various embodiments of the present disclosure.

FIG. 7A is a fluoroscopic image of a patient's chest with minimally invasive diagnostic/therapeutic catheters therein with real and false fiducials identified by an algorithm, consistent with various embodiments of the present disclosure.

FIG. 7B is the fluoroscopic image of FIG. 7A after filtering, with only real fiducials identified, consistent with various embodiments of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION OF EMBODIMENTS

The instant disclosure relates to systems and methods for registering one or more images of an anatomical region of a patient in a coordinate system of a medical system, such as a medical device navigation system. In particular, detection of fiducial markers in the presence of catheters and other medical devices in the one or more images. Details of the various embodiments of the present disclosure are described below with specific reference to the figures.

In various embodiments, a medical navigation system, such as an electro-magnetic tracking and navigation system, may provide the ability to present magnetic information obtained from magnetic sensors on a Cathlab's X-ray images (also referred to herein as fluoroscopic images). To facilitate this, projection matrices (also referred to herein as transformation matrices) from the three-dimensional (3D) world onto a two-dimensional (2D) X-ray image may be calculated. The projection matrices may be calculated using a set of radiopaque elements (fiducials) that are visible on the 2D X-ray image, and are also associated with a known position within a magnetic coordinate system, for example. The radiopaque fiducials may be situated on an optic-magnetic registration plate (OMRP) and included as part of an electro-magnetic navigation system consistent with various embodiments of the present disclosure.

Fluoroscopic images captured during clinical intervention and diagnostic exploration often include medical devices such as catheters and sensors—which may contain radiopaque elements that visually appear similar to the OMRP fiducials. The presence of these “false” fiducial-like elements in the fluoroscopic images may lead to reduced fiducial detection accuracy. The fiducial detection accuracy has a significant impact on the accuracy of the calculated projection matrices, and therefore on the accuracy of the medical navigation system as a whole.

One method of illustrating a medical device in an anatomical region of a patient is to superimpose a real-time rendered representation of the medical device onto a group of images of the anatomical region. This method advantageously allows the clinician operating the medical device to view the device's location relative to the anatomical region without exposing the patient and clinician to excessive imaging radiation. The images are captured by an imaging system and a navigation system tracks the position of the medical device substantially in real time. A programmed electronic control unit creates a composite image for display comprising a representation of the tracked medical device superimposed onto the group of images. To create the composite image, the images may first be registered in the coordinate system of the navigation system—i.e., the coordinate system of each image in the group may be reconciled with the coordinate system of the navigation system.

Aspects of the present disclosure are directed to algorithms for robust detection of radiopaque fiducial balls situated on an OMRP in the presence of other, irrelevant, radiopaque elements introduced by medical devices such as catheters and sensors which appear in a fluoroscopic image. The present disclosure detects the OMRP fiducials, while excluding false fiducial-like elements using various methods for extraction and filtering of ball-shaped radiopaque elements from fluoroscopic images. The fiducials on the OMRP plate facilitate the superimposing of real-time rendered representations of the medical device onto a group of images of an anatomical region within which the medical device is operating.

While various embodiments of the present disclosure are directed to fiducial markers such as radiopaque elements which are visible on fluoroscopic images, aspects of the present disclosure may be applied to non-fluoroscopic imaging and are not at all limited to OMRP type fiducials. Instead, aspects of the present disclosure may be applied to various medical imaging techniques including ultrasonography, magnetic resonance imaging, endoscopy, elastography, tactile imaging, thermography, nuclear medicine functional imaging techniques including positron emission tomography and single-photon emission computed tomography, among others (referred to hereinafter as the imaging system which produces the clinical image(s) for image processing in accordance with the present disclosure).

It is to be understood that while various embodiments of the present disclosure are directed to clinical images including fiducial markers of an OMRP, image processing in accordance with the present disclosure may be implemented to clinical images absent fiducial markers such as those within an OMRP. In such embodiments, the fiducials may be, for example, electrodes of a mapping catheter or ablation catheter. Moreover, the image-processing algorithms and filtering techniques disclosed herein may be utilized to detect other fiducial markers such as catheters, wires, other electrodes, leads, vein ostium, lesions, catheters tips, external titanium balls, among any other objects within an image that may be of interest to a clinician. In one specific embodiment, for example, an image-processing system disclosed herein may identify where two catheters cross each other within a clinical image.

U.S. Patent publication no. 2013/0272592, dated 25 Dec. 2011, teaches systems and methods for registration of fluoroscopic images in a coordinate system of a medical system, and is incorporated by reference as though fully disclosed herein.

FIG. 1A illustrates an imaging and navigation system 10 for use in imaging an anatomical region of a patient 12, such as a heart 14, and for determining the position of, and navigating, a medical device 16 within the anatomical region. Device 16 may comprise, for example, an electrophysiological (EP) mapping catheter, an intracardiac echocardiography (ICE) catheter, an ablation catheter, etc. It should be understood, however, that the disclosed system could be used in imaging a variety of anatomical regions other than a heart 14, and in connection with a variety of diagnostic and treatment devices depending on the anatomical region of interest. System 10 includes an imaging system 18 and a medical device navigation system 20. In accordance with the present invention, system 10 also includes a registration system for registering a group of images of the anatomical region of patient 12 in a coordinate system 22 of navigation system 20. The registration system may include one or more objects 24 and an electronic control unit (ECU) 26.

Imaging system 18 is provided to acquire images of heart 14 or another anatomical region of interest, and comprises a clinical imaging system in the illustrated embodiment. Although a clinical imaging system is described in this embodiment, the invention described herein may find use with other types of imaging systems configured to capture a group of images including, for example, but without limitation, computed tomography (CT) imaging systems and three-dimensional radio angiography (3DRA) systems, among others. System 18 may include a C-arm support structure 28, a radiation emitter 30, and a radiation detector 32. The emitter 30 and detector 32 are disposed on opposite ends of support structure 28, and disposed on opposite sides of patient 12 as patient 12 lays on an operation table 34. Emitter 30 and detector 32 define a field of view 36, and are positioned such that the field of view 36 includes the anatomical region of interest as patient 12 lays on operation table 34. Imaging system 18 is configured to capture images of anatomical features and other objects within field of view 36. Support structure 28 may have freedom to rotate about the patient as shown by lines 38, 40. Support structure 28 may also have freedom to slide along lines 42, 44 (i.e. along the cranio-caudal axis of patient 12) and/or along lines 46, 48 (i.e. perpendicular to the cranio-caudal axis of patient 12). Rotational and translational movement of support structure 28 yields corresponding rotational and translational movement of field of view 36. In yet further embodiments, an electrophysiology lab (EP lab) including the imaging and navigation system 10 may account for various other motion freedoms; for example, the operation table 34 may also be capable of translations, source-intensifier distance (“SID”), cranial-caudal rotation, and detector 32 rotation about its axis. These various other motion freedoms may be accounted for using the imaging and navigation systems disclosed herein.

Imaging system 18 may acquire a group of images of an anatomical region of patient 12 by first shifting along lines 42, 44, 46, 48 to place the anatomical region of interest within field of view 36. Second, support structure 28 may rotate radiation emitter 30 and radiation detector 32 about patient 12, keeping the anatomical region within the field of view 36. Imaging system 18 may capture images of the anatomical region as support structure 28 rotates, providing a group of two-dimensional images of the anatomical region from a variety of angles. The group of images may be communicated to ECU 26 for image processing and displaying on display 58. The group of images may comprise a sequence of images taken over a predetermined time period.

Navigation system 20 is provided to determine the position of medical device 16 within the body of patient 12, and to permit a clinician to navigate device 16 within the body. In the illustrated embodiment, system 20 comprises a magnetic navigation system in which magnetic fields are generated in the anatomical region, and position sensors associated with device 16 generate an output that changes responsive to the position of the sensors within the magnetic field. System 20 may comprise, for example, the system offered for sale under the trademark “GMPS” by St. Jude Medical and generally shown and described in, for example, U.S. Pat. Nos. 6,233,476, 7,197,354 and 7,386,339, the entire disclosures of which are incorporated herein by reference as though fully disclosed herein, or the system offered for sale under the trademark “CARTO XP” by Biosense Webster, Inc. and generally shown and described in, for example, U.S. Pat. Nos. 5,391,199, 5,443,489, 5,558,091, 6,498,944, 6,788,967 and 6,690,963, the entire disclosures of which are incorporated herein by reference as though fully disclosed herein. Although a magnetic navigation system is shown in the illustrated embodiment, it should be understood that the invention could find use with a variety of navigation systems including those based on the creation and detection of axes specific electric fields, such as the system offered for sale by St. Jude Medical, under the trademark “ENSITE NAVX.” Navigation system 20 may include a transmitter assembly 50 and one or more position sensors 52 together with an electronic control unit (ECU) 54.

Transmitter assembly 50 is conventional in the art and may include a plurality of coils arranged orthogonally to one another to produce a magnetic field in and/or around the anatomical region of interest. It should be noted that, although transmitter assembly 50 is shown under the body of patient 12, and under table 34 in FIG. 1A, transmitter assembly 50 may be placed in other locations, such as attached to radiation emitter 30 or radiation detector 32—from which the magnetic field generators can project a magnetic field in the anatomical region of interest. In accordance with certain embodiments of the invention, transmitter assembly 50 is within a field of view 36. In some specific embodiments, the radiation emitter 30 is positioned below the patient 12 and the corresponding radiation detector 32 above the patient.

Position sensors 52 are configured to generate an output dependent on the relative position of sensors 52 within the field generated by transmitter assembly 50. The sensors 52 are in a known positional relationship to device 16, and may be attached to medical device 16. In FIG. 1A, position sensor 52 and medical device 16 are shown disposed within a heart 14. The position sensors 52 may be attached to the distal or proximal end of the device 16, or any point in between. As the medical device 16 is guided to, and through, the anatomical region of interest, navigation system 20 determines the location of the position sensor 52 in the generated field, and thus the position of the medical device 16.

ECU 54 is provided to control the generation of magnetic fields by transmitter assembly 50, and to process information received from sensors 52. ECU 54 may comprise a programmable microprocessor or microcontroller or may comprise an application specific integrated circuit (ASIC). The ECU 54 may include a central processing unit (CPU) and an input/output (I/O) interface. The ECU 54, via the I/O interface, may receive a plurality of input signals including signals generated by sensors 52, and generate a plurality of output signals including those used to control transmitter 50. Although ECU 54 is shown as a separate component in the illustrated embodiment, it should be understood that ECU 54 and ECU 26 may be integrated into a single unit.

Objects 24 are provided to permit registration of images captured by imaging system 18 in coordinate system 22 of navigation system 20. Each object 24 may comprise, for example, one or more fiducial markers. The objects 24 are positioned within a field of view 36 of the imaging system 18. The objects 24 are also either located at a known position within the coordinate system 22, or are connected to a sensor that can provide a position within the coordinate system 22. In the illustrated embodiment, for example, the objects 24 are placed on a component of the navigation system 20 that is within field of view 36, such as transmitter 50. Alternatively, the objects 24 may be placed at a fixed distance from a component, e.g. the transmitter 50, of the navigation system 20, but still within the field of view 36. The objects 24 may also be affixed to a distal end portion of a catheter that is disposed within the field of view 36, or to a body portion of the patient within field of view 36. Alternatively, the objects 24 may include a sensor (not shown) similar to sensor 52 that is locatable in the navigation system 20. The objects 24 may remain in the same known position within the coordinate system 22 over time or may move between different known positions in the coordinate system 22 (e.g., where a subsequent position has a known relationship to a prior known position within the coordinate system 22). In the case of an object 24 that includes a plurality of fiducial markers, the markers may be arranged in a predetermined pattern or otherwise in a manner where the markers have a known relationship to one another. The markers may also have different degrees of radiopacity.

Aspects of the present disclosure are directed to registering an image of image system 18 in coordinate system 22 of navigation system 20 once at the beginning of the procedure, at various stages, or near continuously depending on the needs of a clinician.

Referring to FIG. 1B, in another embodiment of the present disclosure, the registration system includes means 56 for moving fiducial marker 25 between a position disposed within the field of view 36 (such that fiducial marker 25 is in a first state and visible in images captured by imaging system 18) and another position disposed outside the field of view 36 (such that fiducial marker 25 is in a second state and invisible in images captured by imaging system 18). The moving means 56 may comprise any conventional mechanical apparatus that moves the fiducial marker 25 between positions by linear, rotational or other motion (e.g., a linear actuator or a motor driven table) operating under the control of ECU 26. In various embodiments, fiducial marker 25 may include one or more objects 24. In such embodiments, the objects 24 may be spatially coupled to one another via an OMRP thereby limiting independent movement of the objects 24 relative to one another.

In yet another embodiment of the invention, objects 24 are configured with a relatively low intensity and/or a relatively gradual variation in intensity such that objects 24 are substantially invisible to the human eye in the images generated by imaging system 18, but are detectable through image processing by ECU 26 as described herein below. The objects 24 may, for example be relatively thin or may have a low, but existent, radiopacity. In one embodiment, the objects 24 are made from an aluminum foil or aluminum sheet. It should be understood, however, that the objects 24 may be made from a variety of metals, metal alloys or other materials having at least a minimal level of radiopacity. In another embodiment, the radiopacity of the object 24 gradually increases or decreases moving from one point on object 24 to another point on the object 24 (e.g., the radiopacity is greater near a center of an object 24, but gradually decreases moving away from the center of an object 24) taking advantage of the fact that the human eye has difficulty in detecting relatively small changes. The objects 24 may assume any of a variety of sizes and shapes.

ECU 26 is provided for processing images generated by imaging system 18 and registering the images in coordinate system 22 based on the appearance of objects 24 in the images. ECU 26 may also control the operation of medical device 16, the imaging system 18, navigation system 20 and/or a display 58. The ECU 26 may comprise a programmable microprocessor or microcontroller or may comprise an application specific integrated circuit (ASIC). The ECU 26 may include a central processing unit (CPU) and an input/output (I/O) interface through which the ECU 26 may receive a plurality of input signals, including signals generated by the medical device 16, the imaging system 18, and the navigation system 20. Moreover, the ECU 26 may generate a plurality of output signals including those used to control the medical device 16, the imaging system 18, the navigation system 20 and the display 58. The ECU 26 may also receive an input signal from an organ monitor (not shown), such as an ECG monitor, and sort or segregate images from the imaging system 18 based on a timing signal of a monitored organ. For example, the ECU 26 may sort images based on the phase of the patient's cardiac cycle at which each image was collected, as more fully described in, for example, U.S. Pat. No. 7,697,973 which is hereby incorporated by reference in its entirety as though fully disclosed herein.

In accordance with one embodiment of the present disclosure, ECU 26 is configured with appropriate programming instructions or code (i.e., software) to perform several steps in a method for registering a group of images of a patient's heart 14, or another anatomical region of patient 12 in coordinate system 22 of navigation system 20.

Referring now to FIG. 1C, aspects of the present disclosure are directed to methods of superimposing an image of a medical device onto a clinical image. In such an embodiment, a first step 88 includes collecting a plurality of images from a group of images generated by an imaging system 18. The group of collected images includes a region containing one or more objects 24 that is in a known position in a coordinate system. Depending on the intensity of the objects and the processing capabilities of an ECU, the plurality of images collected by the ECU may comprise the entire group of images or a subset of the group of images. Before an actual image location may be determined, fiducial detection and background filtering 89 may be conducted to identify and remove false fiducials that may result in erroneous location determination. The fiducial detection and background filtering 89 both filters out false fiducials, while also enhancing true fiducials within the group of images. Once the true fiducials have been identified, the method may continue with determining an actual image location 90 for the fiducials (also referred to as objects, and fiducial markers) in each of the images. The ECU, responsive to a summation of image data from each image, may identify a potential image location for the fiducials in each of the images. The ECU may use conventional algorithms in summation of the image data including summing and/or averaging the data in order to eliminate noise in the data, and to determine the actual image location of the fiducials in each of the plurality of images.

The method may continue with the creation of a transformation model responsive to the actual image location of the objects in the plurality of images and the known position of the objects in the coordinate system, step 94. Step 96 registers the group of images in the coordinate system 22 using the transformation model. Finally, step 98 superimposes an image of a medical device onto each image in the group of images responsive to the position of the medical device within the coordinate system.

FIG. 1D is a flow diagram 100 for fiducial detection and background filtering, which may take place in step 89 with respect to FIG. 1C, consistent with various embodiments of the present disclosure. The algorithms described in more detail below (and the results of which are presented in FIGS. 2-7) are discussed in reference to ball-shaped fiducials. It is to be understood however that the fiducial detection and background filtering process generally, as well as the specific algorithms therefore, are readily amenable to various other shapes and fiducial configurations. For example, other fiducial arrangements may include elongated fiducials such as lines and rods. Since catheters and other medical tools may present rod-like shapes on a clinical image, aspects of the present disclosure are also directed to filtering out false fiducials related to such catheters and medical tools (see, e.g., FIGS. 6A-6B and discussion thereof).

The various fiducial detection and background filtering algorithms disclosed herein may be applied to an input X-ray image (also referred to as a fluoroscopic image or clinical image), a group of X-ray images, and/or to an averaged X-ray image obtained from a sequence of X-ray images. The fiducial detection and background filtering algorithms may also be applied to ultrasonography, magnetic resonance imaging, endoscopy, elastography, tactile imaging, thermography, nuclear medicine functional imaging techniques including positron emission tomography and single-photon emission computed tomography, among others.

After an input image 101 is received by processor circuitry, also referred to as an ECU (see, FIG. 2A), fiducial enhancement 102 may be undertaken. The fiducial enhancement 102 may utilize an enhancing filter, or other similar filtering technique/algorithm, to improve the appearance of the fiducial elements on the x-ray image (see, FIG. 2B). In various embodiments, a magnetic localization system may utilize fiducials which are small metallic balls that appear as dark spots on the x-ray images. One fiducial implementation utilizes OMRP with a known pattern of fiducials. In some embodiments, the pattern of fiducials may be a uniform or non-uniform pattern. Where the fiducials are in a non-uniform pattern, low error-rate identification of fiducials (as disclosed herein) may be beneficial. Fiducial enhancement 102 may utilize one or more algorithms/filters to enhance the appearance of the fiducial elements over various other features in the image. Some examples of algorithms which may be used to facilitate fiducial enhancement 102 include Laplacian of Gaussian (“LoG”), difference of Gaussians (“DoG”), Hessian filter, steerable filters, and non-linear diffusion-reaction filters (e.g., Beltrami based filters).

OMRPs may also utilize fiducials including radiopaque lines and/or small rods. Similar to ball-shaped fiducials, detection of rod-shaped fiducials may also result in false fiducial detection in prior art systems due to the similarities in shape with catheters, for example. These false rod-shaped fiducials may also utilize the fiducial detection and background filtering method in FIG. 1D to remove such false fiducials.

Several specific embodiments for fiducial enhancement 102 in a clinical image are disclosed below.

In one specific embodiment, a LoG algorithm is used. The LoG is an operator based algorithm, where the operator is based on a second order derivative—ΔI=Iₓₓ+I_{yy}. While the LoG algorithm may be used for “blob” detection, the LoG algorithm is often sensitive to noise. The LoG may be approximated by a DoG at different scales, thus leading to a more efficient implementation of this operator.

In another specific embodiment of fiducial enhancement 102 within a clinical image, a Hessian filter may be used for the automated detection of blob-like structures. Based on an eigenvalues, multiscale analysis of the Hessian matrix, a local pattern may be identified and categorized to belong to plate-like, line-like or blob-like structures. The Hessian of the image for each pixel in the image is:

$$H = ( \begin{matrix}{I}_{xx}^{σ} & {I}_{xy}^{σ} \\ {I}_{xy}^{σ} & {I}_{yy}^{σ}\end{matrix} ) ,$$

where Iₓₓ^{σ}, I_{xy}^{σ}, I_{yy}^{σ} are the smoothed, second order derivatives of the image. The image is smoothed out by a Gaussian sigma parameter (Iₓₓ^{σ}=Iₓₓ*G_{σ}). The eigenvalues λ₁^{σ} and λ₂^{σ} are then sorted (|λ₁^{σ}|<|λ₂^{σ}|). Local structures (by object scale), and shape discrimination (by analysis of the eigenvalues of the Hessian matrix) may then be integrated into a common response measure. The enhancing blobs multiscale response is:

$$B ( {λ}^{σ} ) = {max}_{σ} ( \vert {λ}_{1}^{σ} \vert ) .$$

The enhancing line structure response is:

$$L ( {λ}^{σ} ) = {max}_{σ} ( \vert {λ}_{2}^{σ} \vert ) .$$

In some embodiments for fiducial enhancement 102 in a clinical image, a Beltrami filter may be used. In one specific embodiment, the Beltrami filter is a nonlinear diffusion reaction filter designed for filtering image noise while preserving edges of the image. If a reaction term is added to a diffusion term, contrast enhancement of fiducials balls within the image may be achieved. The processed image output by fiducial enhancement 102 may be seen as an asymptotic state of the partial differential equation based on an evolutionary model. One example evolutionary model is:

Iₜ=Δ_{g}I+ƒ(I),

where Δ_{g} is a Laplace-Beltrami operator (e.g., an extension of a Laplacian operator from flat space to manifolds), and the function ƒ is the reaction term. The reaction term function selection may vary based on a variety of factors, for example, image processing application, and post-processing requirements.

Referring back to FIG. 1D, background removal 103 may be applied to the image to reduce the influence of a patient's heart and other background shadowing within the clinical image. Clinical images may often include large image areas including dark regions (i.e. shadows). Common examples of features that cause such darker regions include bones (e.g., in clinical images of a patient's chest, ribs and spines are often visible as dark gradients), and the patient's cardiac muscle. These dark regions reduce the contrast of fiducials in the image. Accordingly, existing systems are more likely to identify false fiducials due to the minimal contrast variations between true fiducials, dark regions associated with physical features of the patient, as well as medical devices within the clinical image. Background removal 103 increases the contrast within the image between fiducial markers and non-fiducial features. Background removal 103 may utilize various algorithms, including, for example, Morphological filters and/or Median filters. The Median and Morphological filters can eliminate large objects (such as shadows) on the image, while preserving smaller objects including the fiducials markers.

After background removal 103 is complete, three parallel filtering paths are executed. The first filtering path 120 is dedicated to identifying each of the fiducials (also referred to as centroids, objects, and fiducial markers) within the clinical image. The second filtering path 130 identifies, enhances, and filters any catheters within the image. The third filtering path 140 identifies, enhances, and filters out electrodes (e.g., catheter electrodes for electrophysiology mapping and/or ablation, magnetic localization, etc.—often located at a distal end of a catheter shaft).

Each of the three parallel filtering paths, 120, 130, and 140, may filter fiducial candidate components based on their respective size/shape. For example, the first filter path 120 eliminates components that are larger or smaller in size than a typical fiducial. While the first filter path 120 will select true fiducials based on the size/shape, smaller components, such as those generated by image noise, may be ignored by the first filtering path 120.

The first filtering path 120 may include Adaptive Thresholding 104 (also referred to as binary map computing), after background removal 103. A binary map may be computed by thresholding the output image, from background removal 103, to generate a binary, connected components image. The components correspond to the position and size of the dark regions captured in the image (see, e.g., FIG. 7A—702₁₋₂ and 725_{1-N}). Some dark regions are generated from OMRP fiducials, and others originate from false fiducials. Some examples of false fiducials may include catheters, catheter electrodes, a patient reference sensor (“PRS”) for a magnetic localization system, physician tools, guide wires, other medical devices that might be exposed on the images, image noise, other image processing artefacts, and contrast agents which may lead to the appearance of false fiducials (e.g., as a result of contrast buildup at specific locations of the image).

The first filtering path 120 further includes component filter 105. Component filter 105 eliminates components that are larger or smaller in size than the typical fiducial size. In some scenarios, image noise can generate small components. However, such image noise generated components are smaller than a typical fiducial component and are accordingly filtered. The component filter 105 further eliminates components that have shapes which deviate considerably from the typical fiducial component. For example, where the OMRP is comprised of ball-shaped fiducials, elongated components would be discarded by component filter 105.

Once all the false fiducials have been filtered by component filter 105, each remaining fiducial is associated with a centroid (e.g., center of mass) that characterizes the exact coordinate of each fiducial, referred to herein as centroid extraction 106. Centroids can be extracted as an average of the component, or as a weighted average of the component (i.e. center of mass). Centroids may also be extracted by locating the darkest pixel in the component prior to, or after, applying some smoothing to overcome image noise.

A second filtering path 130 includes enhancement of catheter candidates 107. Specifically, catheter enhancement 107 utilizes one or more algorithms to enhance the appearance of catheter candidates over various other miscellaneous features in the image. By enhancing the catheter candidates, the contrast variation between the catheter candidates, image background, and miscellaneous other features is greatly increased. Catheter enhancement 107 may utilize various algorithms, including an enhancing filter, or other similar filtering technique/algorithm, to improve the appearance of the catheter elements on the x-ray image. Some examples of algorithms which may be used to facilitate catheter enhancement 107 include Laplacian of Gaussian (“LoG”), difference of Gaussians (“DoG”), Hessian filter, steerable filters, and non-linear diffusion-reaction filters. In more specific embodiments, the catheter enhancement 107 may be accomplished using a diffusion-reaction filter. The increased contrast between the catheter, image background, and miscellaneous other features facilitates the filtering of catheter candidates 108 from the image—the elimination of these catheter candidates from the image further eliminates the likelihood of false fiducials on, or in proximity to, medical devices such as catheters and sensors.

As can be seen in, for example, FIG. 2A, catheters 220A-B (i.e. a double edge object with varying contrast from the surrounding background) appear as ridges on the image 200. Catheter enhancement 107 may be applied using a Hessian filter which produces a strong response to elongated structures, such as the catheters 220A-B, and also other elongated structures such as guidewires and leads. Other ridge-enhancing filters that were found to be useful for catheter enhancement 107, and that produced substantially equivalent results to the Hessian filter include: Frangi filter, Oriented filters, and any other ridge-enhancing filter(s).

In some specific embodiments, filtering catheter candidates 108 includes thresholding the image from the previous step 107, to create a binary map where the elongated structures are present as large components. These large components correspond to elongated tools such as catheters and other medical devices within the image. While the present embodiment implements an adaptive threshold, other embodiments may implement a constant threshold to filter the catheter candidates.

In a second filtering path 130, centroids detected in proximity to the catheters within the image may be extracted as an average of the component, or as a weighted average of the component (i.e. center of mass). The centroids may also be extracted by locating the darkest pixel in the component prior to, or after, applying some smoothing to overcome image noise. The centroids detected in proximity to, or on the detected catheters, may then be excluded as fiducial candidates.

A third filtering path 140 includes enhancement of electrode candidates 109. Specifically, electrode enhancement 109 utilizes one or more algorithms to enhance the appearance of electrode candidates over various other miscellaneous features in the image. False fiducial candidates may originate from catheter electrodes found on catheters (e.g., diagnostic and therapeutic catheters) and sensors (e.g., patient reference sensor). By enhancing the electrode candidates, the contrast between the electrode candidates, image background, and miscellaneous other features is greatly increased. Electrode Enhancement 109 may utilize various algorithms, including an enhancing filter, or other similar filtering technique/algorithm, to improve the contrast of the electrode elements in the x-ray image. Some examples of algorithms which may be used to facilitate electrode enhancement 109 include Laplacian of Gaussian (“LoG”), difference of Gaussians (“DoG”), Hessian filter, steerable filters, and non-linear diffusion-reaction filters. In more specific embodiments, the electrode enhancement 109 may be accomplished using a diffusion-reaction filter. The increased contrast between the electrode(s), image background, and miscellaneous other features facilitates the filtering of electrode candidates 110 from the image—the elimination of these electrode candidates from the image further eliminates the likelihood of false fiducials on, or in proximity to, medical devices such as electrodes found on catheters and sensors.

In some specific embodiments, filter electrode candidates 110 includes thresholding the image from the previous step, 109, to create a binary map where the elongated structures are present as large components. These large components correspond to elongated tools such as catheters and other medical devices within the image. While the present embodiment implements an adaptive threshold, other embodiments may implement a constant threshold to filter the electrode candidates.

In various embodiments, consistent with the present disclosure, electrode enhancement 109 may utilize one or more scales meant to enhance ball-shaped fiducials of various sizes. As an example, a Hessian filter may be used with various scales to implement such electrode enhancement. Two different variants of the Hessian filter may be used to enhance the outer edges of the ball-shaped electrodes.

During the step of filter electrode candidates 110, binary maps may be produced containing components originating from background removal 103 and electrode enhancement 109. The binary maps may be applied by an ‘AND’ operator, or by a fusion method. The filter electrode candidates step 110 produces an output image where the remaining connected components define fiducial components.

The step of filter electrode candidates 110 may exclude small and large components based on a size threshold. The remaining components, from the electrode enhancement step 109 may then be clustered based on a threshold distance. The large clusters contain mostly catheter electrodes, while the fiducials (which are sparse) stay isolated. Finally, the fiducial candidates which are located below a threshold distance from the large clusters, formed in the previous step, are excluded.

Once the image processing, including the parallel image processing conducted by each of three parallel filtering paths, 120, 130, and 140 for the image is complete, final selection of fiducials 111 may be completed. As the three parallel filtering paths, 120, 130, and 140 have removed the false fiducials associated with catheters and electrodes, the only remaining fiducials will be the true fiducials enhanced by first filter path 120. Accordingly, the remaining fiducials are selected, and the system utilizes the remaining fiducials to determine a transformation model that associates a first Cartesian coordinate system of a clinical image with a second Cartesian coordinate system of a navigation system.

FIG. 2A is a clinical image 200 of a patient's chest with 3 minimally invasive diagnostic/therapeutic catheters therein, consistent with various embodiments of the present disclosure. As shown in FIG. 2A, exploratory catheters 210_{A-B} are extending through a patient's vasculature system toward a cardiac muscle, and an electrophysiology loop catheter 205 has been extended through the vasculature of the patient and into the cardiac muscle. The background of the clinical image contains various shaded elements indicative of portions of the patient's body including ribs, vertebra, and the cardiac muscle itself. The shading of the ribs, vertebra, and heart limit the contrast between the background image and the various catheters 205 and 210_{A-B}, and catheter shafts 220_{A-B}.

This lack of contrast is problematic where image processing is utilized to identify fiducials 202₁₋₂ in the clinical image. It has been discovered that the use of an OMRP including fiducials, where the OMRP is statically positioned within the operating suite (often below the patient, e.g., within/underneath the operating table), facilitates the association of a first Cartesian coordinate system of a clinical image with a second Cartesian coordinate system of a navigation system. Many clinical imaging systems include a C-arm capable of multi-axis rotation to facilitate various images of a patient. To reconcile the first Cartesian coordinate system of a clinical image, which changes depending on the C-arm orientation and translation for an image (among other factors such as operating table translation/rotation, detector rotation about its axis, and SID), with the second Cartesian coordinate system of the navigation system, the OMRP may be placed within the clinical image during exposure. The fiducials on the OMRP facilitate reconciling the unique coordinate systems as the position of the OMRP (which appears on the clinical image) is known relative to the navigation system. Based on a transformation model and tracking data from the navigation system, an image of a tracked object (e.g., catheter) may be overlaid on the clinical image.

To facilitate autonomous reconciling of the unique coordinate systems (e.g., a first Cartesian coordinate system of a clinical imaging system, and a second Cartesian coordinate system of a navigation system), an image processing system may be capable of accurately identifying the fiducial elements within the clinical image without detecting false fiducials. Once the fiducial elements are detected, a transformation model may be determined to properly superimpose the determined location of objects tracked by the navigation system onto the clinical image. If false fiducials are detected by the image processing system and relied on to determine the transformation model, the reconciliation of the coordinate systems will be inaccurate—thereby creating an error in the super-positioning of the navigation system information on the clinical image.

As shown in FIG. 2A, the lack of contrast between the background of clinical image 200 and the various objects (e.g., various catheters 205 and 210_{A-B}, and catheter shafts 220_{A-B}) makes it difficult for an image processing system to differentiate the various objects and fiducials 202₁₋₂ from the background—leading to an increased error rate in the automation of fiducial identification within the clinical image 200.

FIG. 2B is a filtered version of the clinical image 200 of FIG. 2A where fiducials 202₁₋₂ in the image 201 have been enhanced, consistent with various embodiments of the present disclosure. While a contrast ratio between the catheter shafts 220_{A-B} and false fiducials (e.g., electrodes 215_{1-N} on various catheters 205 and 210_{A-B}) remains the same, the contrast ratio between the fiducials 202₁₋₂ and background objects of the clinical image 201 is improved. Some examples of algorithms which may be used to facilitate fiducial enhancement include Laplacian of Gaussian (“LoG”), difference of Gaussians (“DoG”), Hessian filter, steerable filters, and non-linear diffusion-reaction filters (e.g., Beltrami based filters).

FIG. 3 is a clinical image 300 of a patient's chest with minimally invasive diagnostic/therapeutic catheters therein, the clinical image 300 is filtered with two filters. The first filter is a non-linear filter (Beltrami) that enhances ball-shaped fiducials, as well as false fiducials such as electrodes 315_{1-N} on various catheters 305 and 310_{A-B}. The second filter is applied to remove a background image (e.g., portions of an image that may include large scale elements such as ribs, shadows, bones, cardiac muscle, etc., while excluding small scale elements such as fiducials, catheters, and electrodes), consistent with various embodiments of the present disclosure. By removing the background details from the image, an image processing system further increases a contrast ratio between fiducials 302₁₋₂ and the electrodes 315_{1-N}, and a background image (which has all but been removed). The background removal step mostly removes large-scale details such as ribs, shadows, bones, cardiac muscle, etc. from the image 300.

FIG. 4 is a clinical image 400 of a patient's chest with minimally invasive diagnostic/therapeutic catheters therein. With an input of the image 300 of FIG. 3, an image processing system further filters the image with a Hessian filter to enhance fiducials 402₁₋₂ in the image 400. The Hessian filter removes the remaining artefacting from the catheter shafts 320_{A-B} in image 300. After application of the Hessian filter to the image 300, the remaining objects within the image 400 are fiducial markers 402₁₋₂ and false fiducials (e.g., electrodes 415_{1-N} on various catheters 405 and 410_{A-B}).

In FIG. 5, the clinical image 400 of FIG. 4 is further processed using geodesic distance to highlight clustered, false fiducial elements within the image 500, and to further suppress the background. Geodesic distance takes advantage of the close proximity relationship of false fiducials found on catheters. Specifically, electrodes 515_{1-N} on the various catheters 505 and 510_{A-B} within image 500 are often clustered near distal tips of the catheters for facilitating, for example, therapy and/or localization at the distal tip of the catheter. As the true fiducials in the image 400 have much larger spacing then the false fiducials, fiducial markers 402₁₋₂ do not appear in the processed image 500. Accordingly, an image processing system may deduce from a comparison of image 400 and image 500 the location of the true fiducial markers 402₁₋₂. As discussed in more detail above, based on the location of the true fiducials, the coordinate systems of a navigation system and the clinical image 500 may be reconciled using a transformation model.

FIG. 6A is a clinical image 600 of a patient's chest with minimally invasive diagnostic/therapeutic catheters 610_{A-B} and catheter shafts 620_{A-B} therein. The image 600 further includes wire-bundles 622, and a patient reference sensor 621 (“PRS”) located on a patient's chest. As in image 200 of FIG. 2A, a background of the image 600 includes various shaded elements indicative of portions of the patient's body including ribs, vertebra, and the cardiac muscle itself The shading of the ribs, vertebra, and heart limits the contrast between the image background, fiducial markers 602₁₋₂, and catheter electrodes 615_{1-N}, among other features in the image 600. The image processing system may also be prone to falsely identifying the PRS 621 and/or intersections of wires within the wire-bundles 622 as fiducial markers. Accordingly, the image processing system may implement filtering on image 600 to remove such false fiducial inducing objects.

FIG. 6B shows a clinical image 601 after adaptive thresholding and a Hessian filter have been applied to the original clinical image 600, as shown in FIG. 6A. The combination of the adaptive thresholding and the Hessian filter causes the dark segments associated with catheters 610_{A-B} and wire-bundles 622 within the original image 600 to appear as connected components. Only a small artefact of the PRS 621 remains. An image processing system may then deduce from a comparison of images 600 and 601 the location of true fiducial markers 602₁₋₂.

FIG. 7A is a clinical image 700 of a patient's chest with minimally invasive diagnostic/therapeutic catheters 705 and 710_{A-B} extending through a cardiovascular system of the patient. In the image 700 of FIG. 7A, true and false fiducials, 702₁₋₂ and 725_{1-N}, respectively, have been identified by an image processing system, but the image 700 has not been filtered in accordance with the present disclosure. Accordingly, the displayed image 700 has been augmented to overlay highlights on top of all the true fiducials 702₁₋₂, and false fiducials 725_{1-N} associated with electrodes of a catheter 705. Such a result would greatly detriment the accuracy of a transformation model used to reconcile coordinate systems of a navigation system and a clinical imaging system.

FIG. 7B shows the clinical image 700 of FIG. 7A after filtering, consistent with various aspects of the present disclosure. The filtering conducted by an image processing system, centroid extraction, identifies the false fiducials 725_{1-N} associated with catheter 705, and augments the display to overlay highlights on top of only the true fiducials 702₁₋₂.

While the displays shown in FIGS. 7A-B are intended to visually show automated identification of fiducial markers by the image processing system, such an augmented display may be utilized in some embodiments to further verify the accuracy of the image processing system's selection of fiducials. When a new transformation model is required to reconcile the coordinate systems of a navigation system and a clinical imaging system, the image processing system may implement the various filtering schemes disclosed herein and display an augmented image similar to FIGS. 7A-B to a clinician to facilitate human intervention in the automated process. In such an embodiment, the user would verify the accuracy of the image processing systems identification of fiducials, or amend the selections to correct for errors.

In some specific embodiments, the image processing system, to facilitate improved accuracy, may split each of the clinical images into one or more sub-frames. In such an embodiment, the clinical image may be divided into the one or more sub-frames based on positioning of potential fiducial markers therein. In specific embodiments, each sub-frame will have only one potential fiducial marker, or a portion of the potential fiducial markers. Each sub-frame may then be individually processed using the various image processing, filtering, and enhancing techniques disclosed herein to identify the true fiducial markers and to filter out any false fiducial markers within the sub-frames. Once all the sub-frames of the image have been processed, the image may be re-compiled for display to a clinician, for example.

Some specific filtering methods of the present disclosure include removing overlays from the clinical image. Overlays are additional symbols that are not in the native clinical image, but are added by the clinical imaging system. For example, an overlay may include fixed and/or changing symbols. Filtering methods disclosed herein may remove such overlays from the image prior to identifying fiducial markers, which further reduces the likelihood of identifying false fiducials.

Although several embodiments have been described above with a certain degree of particularity, those skilled in the art could make numerous alterations to the disclosed embodiments without departing from the spirit of the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Based upon the above discussion and illustrations, those skilled in the art will readily recognize that various modifications and changes may be made to the various embodiments without strictly following the exemplary embodiments and applications illustrated and described herein. For example, one or more of the imaging processing filters disclosed above may be used in series and/or in parallel to provide additional fiducial marker results to cross-check another result that utilizes a different filter and/or combination of filters. Such modifications do not depart from the true spirit and scope of various aspects of the invention, including aspects set forth in the claims.

Various modules or other circuits may be implemented to carry out one or more of the operations and activities described herein and/or shown in the figures. In these contexts, a “module” is a circuit that carries out one or more of these or related operations/activities (e.g., an image processing system). For example, in certain of the above-discussed embodiments, one or more modules are discrete logic circuits or programmable logic circuits configured and arranged for implementing these operations/activities. In certain embodiments, such a programmable circuit is one or more computer circuits programmed to execute a set (or sets) of instructions (and/or configuration data). The instructions (and/or configuration data) can be in the form of firmware or software stored in and accessible from a memory (circuit). As an example, first and second modules include a combination of a CPU hardware-based circuit and a set of instructions in the form of firmware, where the first module includes a first CPU hardware circuit with one set of instructions and the second module includes a second CPU hardware circuit with another set of instructions.

Certain embodiments are directed to a computer program product (e.g., nonvolatile memory device), which includes a machine or computer-readable medium having stored thereon instructions which may be executed by a computer (or other electronic device) to perform these operations/activities.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A method for identifying and locating fiducial markers on a clinical image, the method including:
receiving the clinical image from an imaging system;
processing the clinical image to remove false fiducial markers; and
identifying fiducial markers in the clinical image.

2. The method of claim 1, further including applying an enhancing filter to the clinical image to enhance the appearance of the fiducial markers.

3. The method of claim 2, wherein the enhancing filter may be one or more of the following: Laplacian of Gaussian, difference of Gaussians, Hessian filter, steerable filters, and non-linear diffusion-reaction filters.

4. The method of claim 2, wherein the enhancing filter is a Beltrami-based filter.

5. The method of claim 2, wherein the enhancing filter is a Laplacian of Gaussian filter with an operator based on a second order derivative of the image—ΔI=Iₓₓ+I_{yy}.

6. The method of claim 5, wherein the enhancing filter includes the Laplacian of Gaussian filter and a difference of Gaussians filter.

7. The method of claim 2, wherein the enhancing filter is a Hessian filter that includes:
taking the Hessian of each pixel within the clinical image, the Hessian matrix for each pixel is:
$$H = ( \begin{matrix}{I}_{xx}^{σ} & {I}_{xy}^{σ} \\ {I}_{xy}^{σ} & {I}_{yy}^{σ}\end{matrix} ) ,$$
where Iₓₓ^{σ}, I_{xy}^{σ}, I_{yy}^{σ} are smoothed, second order derivatives of the image;
applying a Gaussian sigma parameter, (Iₓₓ^{σ}=Iₓₓ*G_{σ}), to smooth the image;
performing eigenvalue multi-scale analysis of the Hessian matrix;
sorting eigenvalues λ₁^{σ} and λ₂^{σ}(|λ₁^{σ|<|λ}₂^{σ}|);
sorting local structures by object scale, and shape discrimination by analysis of the eigenvalues of the Hessian matrix;
enhancing a multiscale response of objects with:
$$B ( {λ}^{σ} ) = {max}_{σ} ( \vert {λ}_{1}^{σ} \vert ) ; and$$
enhancing line structure response with:
$$L ( {λ}^{σ} ) = {max}_{σ} ( \vert {λ}_{2}^{σ} \vert ) .$$

8. The method of claim 2, wherein the enhancing filter is a Beltrami-based filter including a nonlinear diffusion reaction filter configured to filter image noise while preserving boundary lines.

9. The method of claim 8, wherein the nonlinear diffusion reaction filter includes a diffusion term, and a reaction term to facilitate contrast enhancement of the fiducials markers within the image.

10. The method of claim 2, wherein the clinical image, after the enhancing filter, is interpreted with an asymptotic state of a partial differential equation based on an evolutionary model.

11. The method of claim 10, wherein the evolutionary model is:
Iₜ=Δ_{g}I+ƒ(I),
where Δ_{g} is a Laplace-Beltrami operator, and the function ƒ is the reaction term.

12. A method to detect medical device electrodes within a clinical image, the method including:
receiving the clinical image from an imaging system;
applying an enhancing filter to the clinical image to enhance the appearance of the electrodes;
removing a background of the clinical image; and
identifying electrodes within the clinical image.

13. The method of claim 12, wherein identifying electrodes within the clinical image includes adaptive thresholding, component filtering based on size and shape, and extraction of the electrodes.

14. The method of claim 12, wherein identifying electrodes within the clinical image includes adaptive thresholding.

15. The method of claim 12, further including filtering the clinical image to remove false fiducials.

16. The method of claim 15, wherein the step of filtering the clinical image to remove false fiducials includes using one or more of the following: a Beltrami-based enhancing filter, a Laplacian of Gaussian enhancing filter with an operator based on a second order derivative of the image, and a Laplacian of Gaussian filter and a difference of Gaussians filter.

17. The method of claim 15, wherein the step of filtering the clinical image to remove false fiducials includes filtering the clinical image with a Hessian filter, filtering with the Hessian filter includes:
taking the Hessian of each pixel within the clinical image, the Hessian matrix for each pixel is:
$$H = ( \begin{matrix}{I}_{xx}^{σ} & {I}_{xy}^{σ} \\ {I}_{xy}^{σ} & {I}_{yy}^{σ}\end{matrix} ) ,$$
where Iₓₓ^{σ}, I_{xy}^{σ}, I_{yy}^{σ} are smoothed, second order derivatives of the image;
applying a Gaussian sigma parameter, (Iₓₓ^{σ}=Iₓₓ*G_{σ}), to smooth the image;
performing eigenvalue multi-scale analysis of the Hessian matrix;
sorting eigenvalues λ₁^{σ} and λ₂^{σ}(|λ₁^{σ}|<|λ₂^{σ}|);
sorting local structures by object scale, and shape discrimination by analysis of the eigenvalues of the Hessian matrix;
enhancing a multiscale response of objects with:
$$B ( {λ}^{σ} ) = {max}_{σ} ( \vert {λ}_{1}^{σ} \vert ) ; and$$
enhancing line structure response with:
$$L ( {λ}^{σ} ) = {max}_{σ} ( \vert {λ}_{2}^{σ} \vert ) .$$

18. A method to detect and distinguish catheters and catheter components from fiducial markers, the method including:
receiving a clinical image from an imaging system;
processing the clinical image to remove the catheters and catheter components from the clinical image; and
identifying fiducial markers in the clinical image.

19. The method of claim 18, further including applying an enhancing filter to the clinical image to enhance the appearance of the fiducial markers.

20. The method of claim 19, wherein the enhancing filter includes one or more of the following: Laplacian of Gaussian, difference of Gaussians, Hessian filter, steerable filters, and non-linear diffusion-reaction filters.

21. A method for superimposing location data of a medical device from a navigation system onto a clinical image, the method including:
receiving the clinical image from a clinical imaging system;
processing the clinical image to remove false fiducial markers;
identifying fiducial markers within the clinical image;
creating a transformation model that reconciles a first coordinate system of the navigation system with a second coordinate system of the clinical imaging system;
determining a location of the medical device in the first coordinate system;
applying the transformation model to the location of the medical device in the first coordinate system to determine the location of the medical device in the second coordinate system; and
superimposing an image of the medical device onto the clinical image based on the known location of the medical device in the second coordinate system.

22. The method of claim 21, wherein the step of processing the clinical image to remove false fiducial markers includes applying an enhancing filter to the clinical image to enhance the appearance of the fiducial markers.

23. The method of claim 22, wherein the enhancing filter may be one or more of the following: Laplacian of Gaussian, difference of Gaussians, Hessian filter, steerable filters, and non-linear diffusion-reaction filters.

24. The method of claim 22, wherein the enhancing filter is a Beltrami-based filter.

25. The method of claim 22, wherein the enhancing filter is a Laplacian of Gaussian filter with an operator based on a second order derivative of the image—ΔI=Iₓₓ+I_{yy}.

26. The method of claim 25, wherein the enhancing filter includes the Laplacian of Gaussian filter and a difference of Gaussians filter.

27. The method of claim 22, wherein the enhancing filter is a Hessian filter that includes:
taking the Hessian of each pixel within the clinical image, the Hessian matrix for each pixel is:
$$H = ( \begin{matrix}{I}_{xx}^{σ} & {I}_{xy}^{σ} \\ {I}_{xy}^{σ} & {I}_{yy}^{σ}\end{matrix} ) ,$$
where Iₓₓ^{σ}, I_{xy}^{σ}, I_{yy}^{σ} are smoothed, second order derivatives of the image;
applying a Gaussian sigma parameter, (Iₓₓ^{σ}=Iₓₓ*G_{σ}), to smooth the image;
performing eigenvalue multi-scale analysis of the Hessian matrix;
sorting eigenvalues λ₁^{σ} and λ₂^{σ (|λ}₁^{σ}|<|λ₂^{σ}|);
sorting local structures by object scale, and shape discrimination by analysis of the eigenvalues of the Hessian matrix;
enhancing a multiscale response of objects with:
$$B ( {λ}^{σ} ) = {max}_{σ} ( \vert {λ}_{1}^{σ} \vert ) ; and$$
enhancing line structure response with:
$$L ( {λ}^{σ} ) = {max}_{σ} ( \vert {λ}_{2}^{σ} \vert ) .$$

28. The method of claim 22, wherein the enhancing filter is a Beltrami-based filter including a nonlinear diffusion reaction filter configured to filter image noise while preserving boundary lines.

29. The method of claim 28, wherein the diffusion reaction filter includes a diffusion term, and a reaction term to facilitate contrast enhancement of the fiducials markers within the image.

30. The method of claim 22, wherein the clinical image, after the enhancing filter, is interpreted with an asymptotic state of a partial differential equation based on an evolutionary model.

31. The method of claim 30, wherein the evolutionary model is:
IₜΔ_{g}I+ƒ(I),
where Δ_{g} is a Laplace-Beltrami operator, and the function ƒ is the reaction term.

32. A method for identification of fiducial markers within a clinical image, the method including:
processing the clinical image to remove false fiducial markers; and
identifying fiducial markers within the clinical image.

33. The method of claim 32, further including enhancing the fiducial markers within the clinical image.

34. The method of claim 32, further including removal of a background within the clinical image.

35. The method of claim 32, wherein the step of processing the clinical image to remove false fiducial markers includes enhancing and filtering a plurality of catheters within the clinical image.

36. The method of claim 32, wherein the step of processing the clinical image to remove false fiducial markers includes enhancing and filtering a plurality of electrodes within the clinical image.

37. The method of claim 32, wherein identifying fiducial markers within the clinical image includes adaptive thresholding, component filtering based on size and shape, and extraction of the fiducial markers.

38. The method of claim 32, wherein identifying fiducial markers within the clinical image includes adaptive thresholding.

39. The method of claim 32, wherein the step of processing the clinical image to remove false fiducial markers includes filtering the clinical image using a Beltrami-based enhancing filter.

40. The method of claim 32, wherein the step of processing the clinical image to remove false fiducial markers includes filtering the clinical image with a Laplacian of Gaussian enhancing filter with an operator based on a second order derivative of the image.

41. The method of claim 32, wherein the step of processing the clinical image to remove false fiducial markers includes filtering the clinical image with a Laplacian of Gaussian filter and a difference of Gaussians filter.

42. The method of claim 32, wherein the step of processing the clinical image to remove false fiducial markers includes filtering the clinical image with a Hessian filter, filtering with the Hessian filter includes:
taking the Hessian of each pixel within the clinical image, the Hessian matrix for each pixel is:
$$H = ( \begin{matrix}{I}_{xx}^{σ} & {I}_{xy}^{σ} \\ {I}_{xy}^{σ} & {I}_{yy}^{σ}\end{matrix} ) ,$$
where Iₓₓ^{σ}, I_{xy}^{σ}, I_{yy}^{σ} are smoothed, second order derivatives of the image;
applying a Gaussian sigma parameter, (Iₓₓ^{σ}=Iₓₓ*G^{σ}), to smooth the image;
performing eigenvalue multi-scale analysis of the Hessian matrix;
sorting eigenvalues λ₁^{σ} and λ₂^{σ} (|λ₁^{σ}|<|λ₂^{σ}|);
sorting local structures by object scale, and shape discrimination by analysis of the eigenvalues of the Hessian matrix;
enhancing a multiscale response of objects with:
$$B ( {λ}^{σ} ) = {max}_{σ} ( \vert {λ}_{1}^{σ} \vert ) ; and$$
enhancing line structure response with:
$$L ( {λ}^{σ} ) = {max}_{σ} ( \vert {λ}_{2}^{σ} \vert ) .$$

43. The method of claim 39, wherein the Beltrami-based filter includes a nonlinear diffusion reaction filter to filter image noise while preserving boundary lines.

44. A navigation system for a cardiovascular catheter, the navigation system comprising:
a cardiovascular catheter including one or more electrodes positioned near a distal tip of the catheter, the electrodes configured and arranged to facilitate localization of the distal tip of the catheter;
an optic-magnetic registration plate including a plurality of fiducial markers;
a clinical imaging system configured and arranged to expose a clinical image including a patient, the fiducial markers, and the cardiovascular catheter in a first coordinate system;
a catheter localization system configured and arranged to detect the position and orientation of the one or more electrodes in a second coordinate system; and
controller circuitry communicatively coupled to the clinical imaging system and the catheter localization system, the controller circuitry configured and arranged to
based on known locations of the fiducial markers in the second coordinate system and the location of the fiducial markers within the clinical image, determine a transformation model between the first and second coordinate systems, and
based on the transformation model, determine positions of the electrodes within the first coordinate system.

45. The navigation system of claim 44, further including a display; the controller circuitry communicatively coupled to the display and further configured and arranged to generate a signal for the display including the clinical image superimposed with a representative image of the catheter based on the determined positions of the electrodes in the first coordinate system.

46. The navigation system of claim 44, wherein the controller circuitry is further configured and arranged to
filter the clinical image to remove false fiducial markers, and
identify fiducial markers within the clinical image.

47. The navigation system of claim 46, wherein the controller circuitry is further configured and arranged to apply an enhancing filter to the clinical image to enhance the appearance of the fiducial markers and the false fiducial markers relative to a background.

48. The navigation system of claim 47, wherein the enhancing filter includes one or more of the following: Laplacian of Gaussian, difference of Gaussians, Hessian filter, steerable filters, and non-linear diffusion-reaction filters.

49. The method of claim 1, wherein the step of identifying fiducial markers in the clinical image includes identifying fiducial markers in a non-uniform pattern.
